(19) United States
(12) Patent Application Publication
(10) Pub. No.: US 2014/0330245 A1
(43) Pub. Date: Nov. 6, 2014

(54) DELIVERY OF A THERAPEUTIC AGENT VIA INTERMITTENT INFUSION

(71) Applicant: RENISHAW PLC, Gloucestershire (GB)

(72) Inventor: Steven GILL, Gloucestershire (GB)

(21) Appl. No.: 14/268,731

(22) Filed: May 2, 2014

(30) Foreign Application Priority Data
May 3, 2013 (GB) 1308035.3

(51) Int. Cl. A61M 5/172

(52) U.S. Cl. CPC A61M 5/172; A61M 5/1723; A61M 2005/14288
USPC 604/503; 604/500; 604/67

(57) ABSTRACT

There is provided a method of reducing or preventing catheter reflux during infusion of a therapeutic agent into a brain of a subject comprising administering the therapeutic agent to a target area of the brain by intermittent infusion at a predetermined interval. There is provided a method of treating a CNS disorder comprising administering a therapeutic agent to a target area of a brain of a subject by intermittent infusion at a predetermined interval. There is also provided a system for reducing or preventing catheter reflux during infusion of a therapeutic agent into a target area of a brain of a subject comprising an infusion device, at least one chronically implanted intracranial catheter, a monitoring device configured to monitor the target area of the brain after each infusion, and a determination unit configured to determine whether a predetermined interval is acceptable.

This application claims the benefit of GB 1308035.3, filed May 3, 2013. The disclosure of the prior application is hereby incorporated by reference herein in its entirety.

### TECHNICAL FIELD

This disclosure is directed to systems and methods for reducing or preventing catheter reflux during infusion of a therapeutic agent into a brain of a subject. Methods of treating CNS disorders are also provided.

### BACKGROUND

Delivery or infusion through an intracranial or neurological catheter involves delivering drugs to the brain parenchyma using micro-catheters and controlled infusion rates to distribute drugs homogenously through the extracellular space, carried by bulk flow. Methods for delivery or infusion through an intracranial or neurological catheter may be used to deliver a wide range of therapeutics for neurological disease that can be targeted to specific brain areas, bypassing the blood brain barrier, and limiting side effects.

Drug distribution by intracranial infusion is achieved by establishing a pressure gradient at the tip of the catheter that is sufficient to drive infusate through the extracellular space, in preference to it refluxing back along the catheter-tissue interface. To distribute therapeutic agents homogenously through large and clinically relevant volumes, the flow rate needs to be close to the maximum flow rate that the brain can safely tolerate. This is because the pressure gradient drops exponentially from the catheter tip and so to achieve bulk flow one has to establish a sufficient pressure gradient up to the boundary of the desired brain volume whilst competing against dynamic extracellular fluid clearance, particularly through the perivascular spaces, which act as peristaltic pumps. Excessive flow rates at the catheter tip will however result in tissue fracturing, and once this has occurred, the fracture will tend to be propagated in preference to distribution through the extracellular space. In addition, high flow rates are associated with increased reflux along the catheter-tissue interface, and the magnitude of this is related to the extent of tissue trauma produced by catheter insertion and to the catheter's external diameter.

Significant adverse effects have been reported from clinical trials which are directly attributable to reflux of infusate into the subarachnoid space, including chemical meningitis, wound dehiscence and spinal root irritation. Therefore, it is desirable to reduce reflux of the infusate.

Reflux can be reduced when infusing into grey matter at flow rates of up to 5 μl/min, by using catheters that have an outside diameter of approximately 0.4 mm or less. When catheters of a larger diameter are employed, they cause greater tissue trauma upon insertion in the annular space around them, and through this low resistance pathway, the infusate will reflux. However, it has also been shown that if a catheter of larger diameter (0.6 mm) is left in situ for sufficient time to allow the tissue to heal, then its tendency to reflux is substantially reduced.

It is also known that the tendency for a catheter to reflux can be reduced by a gradual ramp up of infusion rate, from a baseline infusion, for example 0.5 μl/min, stepwise over 20 minutes, up to 5 μl/min. It is thought that this gradual expansion of the interstitial spaces, under the influence of positive pressure and fluid content, increases the tissues fluid conductivity, while at the same time, the increased tissue pressure may act to improve the tissue seal along the tissue/catheter interface. It is of note that higher flow rates of infusion without reflux can be achieved in white matter than in gray matter (up to 10 μl/min) due to white matter's greater poroelasticity. Moreover, as effective intracranial infusion depends on the use of appropriate catheter designs and infusion parameters, optimized drug delivery systems need to be developed whereas effective vector-mediated gene therapy or RNA interference can be achieved following a single infusion of vector, to practically administer therapeutic agents such as neurotrophic factors to treat neurodegenerative diseases, cytotoxic agents to treat tumors and enzymes to treat conditions such as lysosomal storage diseases, an implantable drug delivery system allowing chronic or intermittent drug delivery is an absolute pre-requisite (see Bienemann, A. et al., The development of an implantable catheter system for chronic or intermittent convection-enhanced delivery, Journal of Neuroscience Methods, 203 (2012) 284-291).

These and other issues are addressed by the present disclosure. It is an object of this disclosure to provide methods and systems that allow chronic intermittent intracranial infusion suitable for use in medical practice and to prevent or reduce reflux when delivering infusate into the brain.

### SUMMARY

In a first embodiment, there is provided a method of reducing or preventing catheter reflux during infusion of a therapeutic agent into a brain of a subject comprising administering the therapeutic agent to a target area of the brain by intermittent infusion at a predetermined interval.

In a another embodiment, there is provided a method of treating a CNS disorder comprising administering a therapeutic agent to a target area of a brain of a subject by intermittent infusion at a predetermined interval.

In another embodiment, there is provided a system for reducing or preventing catheter reflux during infusion of a therapeutic agent into a target area of a brain of a subject comprising an infusion device for administering a therapeutic agent to the target area of the brain; at least one chronically implanted intracranial catheter for delivering the therapeutic agent from the infusion device to the target area; a monitoring device configured to monitor the target area of the brain after each infusion to determine a value of a parameter; a determination unit configured to determine whether a predetermined interval is acceptable based on the value of the parameter, wherein the infusion device administers the therapeutic agent by intermittent infusion at the predetermined interval.

These and other features and advantages of the disclosed systems and methods are described in, or apparent from, the following detailed description of various exemplary embodiments.

### BRIEF DESCRIPTION OF THE DRAWINGS

FIG. 1A illustrates the problem in the conventional art regarding significant reflux at 0 weeks;

FIG. 1B illustrates the problem in the conventional art regarding significant reflux at 2 weeks;

FIG. 1C illustrates the problem in the conventional art regarding significant reflux at 4 weeks;

FIG. 2A illustrates signal change in the putamen with intermittent infusion at 2 weeks;

FIG. 2B illustrates the unexpected benefits achieved from exemplary embodiments of this disclosure;

FIG. 3 illustrates a delivery system according to an embodiment; and

FIG. 4 is a schematic representation of a system according to an embodiment.

### DETAILED DESCRIPTION OF EMBODIMENTS

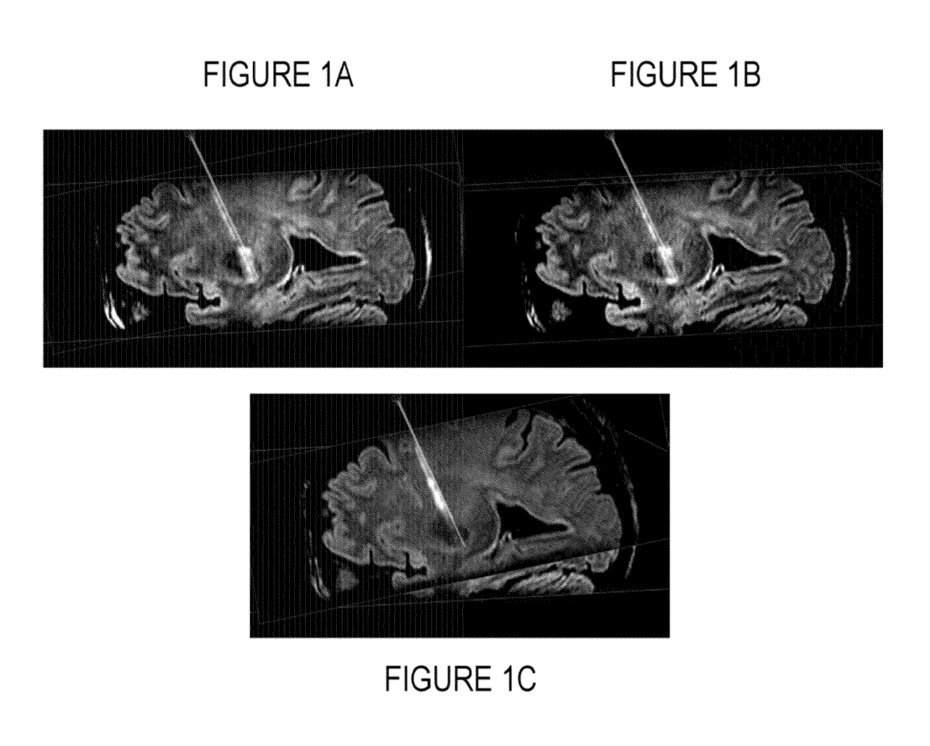

This disclosure is directed to overcoming challenges in the conventional art with systems and methods for reducing or preventing catheter reflux during infusion of a therapeutic agent into a brain of a subject. The problem with conventional infusion techniques for delivering therapeutic agents to the brain is illustrated in FIG. 1 (related art). FIG. 1 shows MRI images of Gd-DTPA and artificial CSF bilaterally infused into the dorsal putamen of a pig via intracranial infusion. Consecutive infusions were made at 2 week intervals for 4 weeks. Imaging was conducted on the pig brain following each infusion at T₀ (week 0) (FIG. 1A), T₁ (week 2) (FIG. 1B), and T₂ (week 4) (FIG. 1C). Compared to infusion 1 at T₀ (FIG. 1A) and T₁ (FIG. 1B), significant reflux can be seen following infusion at T₂ (FIG. 1C). According to systems and methods of this disclosure, the problem of reflux is significantly reduced.

In a first embodiment, there is provided a method of reducing or preventing catheter reflux during infusion of a therapeutic agent into a brain of a subject comprising administering the therapeutic agent to a target area of the brain by intermittent infusion at a predetermined interval. The therapeutic agent can be delivered by intracranial infusion. The therapeutic agent may be administered at a predetermined interval of at least about 21 days. Preferably, the predetermined interval is at least about 28 days. The predetermined interval is not specifically limited by this disclosure and may be at least 35 days, at least 45 days, or at least 6 months. Determining the length of the predetermined interval is relevant in arriving at an appropriate therapy regimen for a patient. A predetermined interval may vary and what is ideal for one patient or circumstance may be different than what is ideal for another patient or circumstance. Infusing therapeutic agent intermittently at predetermined intervals of at least 21 days has unexpectedly been shown to allow brain tissue to heal between each infusion. Tissue healing is one factor to be considered in ascertaining the predetermined interval.

Tissue healing allows the tissue to create an improved seal around the catheter, reducing or preventing reflux of infusate, especially along the tissue/catheter interface. Tissue trauma occurs when a catheter is inserted into a patient. During intracranial infusions fluid is forced under pressure into tissue surrounding the catheter leading to tissue trauma. Reflux is more likely to occur and to be severe when infusing into this damaged tissue. This is particularly problematic when infusing into gray matter of the brain due to the tightly packed cells limiting the interstitial space and the low poroelasticity compared to white matter, making gray matter more vulnerable to damage by infusion. It is an object of the systems and methods of this disclosure to reduce the impact of damaged tissue on reflux.

The systems and methods further comprise determining the amount of healing and whether such healing is sufficient according to the predetermined interval. An intracranial catheter is implanted into the target area of the brain of the subject. After implantation, there is an initial wait time period that is at least 28 days during which healing from the implantation occurs. After the initial wait period expires, a test infusion may be carried out through the implanted intracranial catheter, for example with artificial cerebrospinal fluid (aCSF) or aCSF combined with a tracer, such as Gadolinium. The type of tracer selected is not specifically limited in this embodiment. The tracer may depend on the imaging technique that is used. For a PET, scan a radioisotope is appropriate. For a CT scan, barium would be appropriate. The tracers can be infused in conjunction with a carrier, such as aCSF.

One purpose of the test infusion is to determine a level of reflux before setting the predetermined interval and developing the appropriate therapy regimen. aCSF and Gadolinium can be viewed in an MRI image using any of the imaging or monitoring devices disclosed herein or otherwise known in the art. The target area of the brain is then monitored using an appropriate imaging technique by which the aCSF/tracer is visible. A determination is then made as to whether the observed distribution of the infusion sufficiently resembles a comparator distribution. The determination may be made by a medical professional or by the determination unit 160. The comparator distribution is determined based on optimized in-vitro gel infusions with the intracranial catheter. The determination of whether the observed distribution pattern meets the comparator distribution is based on a sufficient likeness standard. This is a judgment made according to expertise of the medical professional or an algorithmic decision made by a program executed by the determination unit 160. If the observed test infusion distribution pattern is of sufficient likeness to the comparator distribution pattern, then a medical decision is made that sufficient healing has occurred. The predetermined interval is established and the therapy regimen can begin. For example, the predetermined interval could be set at 28 days such that the planned drug infusion would be carried out 28 days after the test infusion.

Other factors to consider in determining the predetermined interval and therapy regimen include volume of the infusate. For example, if the test wait period is 28 days, and the infusion comprises infusing a similar volume to the therapeutic agent infusate, the therapy regimen could be carried out 4 weeks after the test infusion, or immediately after the test infusion if the test infusate is of a smaller volume than the therapeutic agent infusion. Typically the therapeutic agent infusate is about 400 μl (microliters). A test infusate of about 60 μl (microliters) may be sufficient to determine if reflux is likely to occur. As would be understood by one of ordinary skill in the art, the specific drug infusate and test infusate volumes are not particularly limited and may vary based on conditions of the patient, the intracranial catheter system, the type of therapy and the therapeutic agent or test substance infused.

In the event that the test infusion fails the sufficient likeness determination, then it may be determined to wait for a longer period of time, such as 5 weeks or 35 days, before beginning anew with the test infusion and subsequent steps as described herein. The time period is continuously extended until a time period is found in which sufficient healing occurs such that a desired distribution is formed or the catheter implantation is deemed incapable of delivering the therapeutic agent with the required distribution pattern. If an acceptable distribution is found to occur after a certain time period, then therapeutic agent infusions are carried out at predetermined intervals that are equal to or greater than this time period.

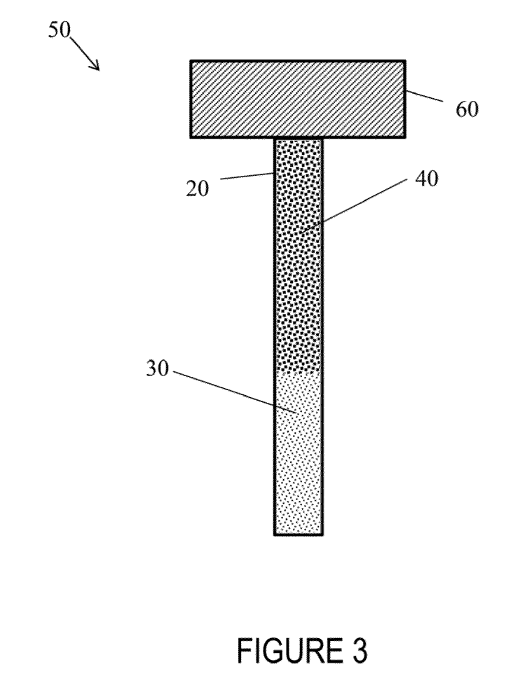

The systems and methods may further include the embodiment illustrated in FIG. 3. FIG. 3 shows a delivery system 50 including a catheter 20 for delivering an infusate and an infusion controller 60 for controlling the amount of infusate delivered through the catheter 20. The catheter 20 may be any of the types of catheters disclosed herein, including recess and step catheters, and is not limited by the type or shape of the catheter shown in FIG. 3. The infusion controller 60 may be a CPU and/or program executable on a CPU, or any controller known in the art suitable for infusing an infusate. The catheter 20 contains a therapeutic agent 40 front-loaded in a catheter 20 with a tracer that uses a lead portion 30, or dead volume, of the therapeutic agent infusate as the test infusion. Preferably, there is a dead volume of about 600 (microliters) of aCSF in front of the therapeutic agent 40. It is known to front load a therapeutic agent in the lead portion 30 with aCSF and to infuse a tracer with aCSF as a carrier, but the systems and methods provide the advantage of front loading a therapeutic agent 40 with lead portion 30 comprising aCSF carrying a tracer. This combination allows one to carry out a test infusion before a drug infusion using the fluid that is retained in the dead volume.

The determination of whether the test infusion distribution pattern is of sufficient likeness to the comparator pattern can be made in near real-time during the infusion. If sufficient likeness is found, the test infusate is front-loaded in the infusate, and the therapeutic agent is infused into the target area of the brain immediately following the test infusate. If the test infusion showed that reflux occurred, then infusion of the drug could be stopped. In the event the infusion is stopped, the system and method further comprises means for determining whether the therapeutic agent in the dead volume should be infused or left in the dead volume of the intracranial catheter assembly. Preferably, it is infused. If there was no reflux after infusion of the dead volume of aCSF, then the therapeutic agent infusion could proceed as normal. The dead volume of aCSF may also comprise a tracer for enhancing monitoring capability, as discussed herein.

The systems and methods may further include inserting a catheter(s) into the brain of a subject and leaving it in place for at least 21 days, more preferably at least 28 days, before any infusions are initiated. This has been shown, unexpectedly, to allow time for the tissue to heal following insertion of the catheter, thereby creating a good seal around the catheter to reduce or prevent reflux along the tissue/catheter interface. The interval before the first infusion is not specifically limited by this disclosure and may be at least 35 days, at least 45 days, or at least 6 months, and may depend on the profile of specific a patient or circumstance. The disclosed intervals result in improved volumes of distribution when infusate is delivered into healed tissue.

Too much healing of the tissue of the target area of the brain is another factor considered according to the systems and methods. For example, even though drugs may be delivered over periods that are greater than 6 weeks, it may be desirable to maintain at least the initial infusions within a 6 weeks period. It has been shown that complete healing of the brain may occur within 6 weeks, and thus disrupt infusion because the interstitial cellular spaces that were opened up by a previous infusion within the target volume are likely to have healed within 6 weeks. Therefore, it is advantageous to base the predetermined interval on this factor as well in determining how to carry out a subsequent infusions before these passageways have fully healed. This reduces the work that needs to be done by the fluid in reopening these passageways and forming new passageways, while ensuring that the time period is sufficient for sufficient healing to have occurred around the recess step to prevent reflux. This provides the advantage of reducing the pressure around the tip of the catheter during infusion if the infusate can escape through preformed cellular spaces such that less damage is caused to the tissue in this region than would be the case if the brain had completely healed. Establishing the appropriate predetermined interval based on all of the circumstances described herein is critical to establishing the most effective treatment regimen.

The method may further include delivering the therapeutic agent into gray matter of the brain such as the cerebral cortex and/or the putamen. The area of the brain targeted by infusions according to the methods and systems of this disclosure may also include, for example, the thalamus, hypothalamus, hippocampus or amygdala. The methods and systems according to this disclosure are not particularly limited by the region of the brain targeted, or the location in the CNS, except that the predetermined interval and pre-infusion interval, may be a function of the region of the brain targeted, and the monitoring of the region of the brain targeted during the infusion regimen.

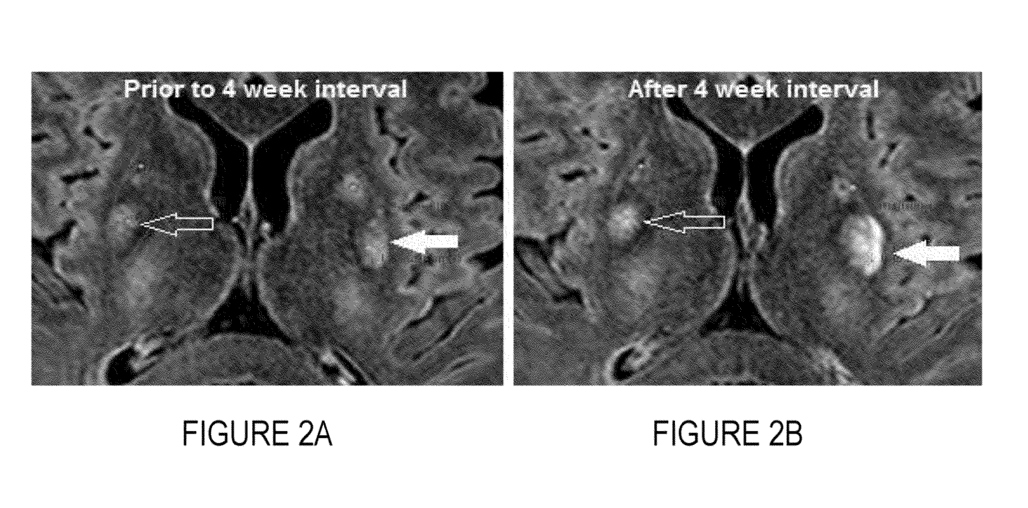

FIG. 2 illustrates an axial cross section of the brain (T2 weighted MRI) of a patient receiving bilateral infusions into the putamen via four catheters. FIG. 2A shows high signal change in both putamen after infusions on a 2 week infusion interval. The volume of distribution is seen to be less than that shown in FIG. 2B where the infusion interval was 28 days, or 4 weeks. Higher diffusion of the therapeutic agent leads to better therapy regimens. The brain tissue healed during the 28 day interval because the interval caused an improved tissue seal around the catheter.

While the minimum predetermined interval between infusions should be at least about 21 days the maximum interval between infusions may be determined by a number of factors or single factor. These factors (e.g., blood pressure) may be measured by a system or device for measuring or capturing values (e.g., 120/80) associated with the factors, or parameters. For example, the clearance rate or biological half-life of the therapeutic agent being administered in a target area of the brain. The minimum concentration of the therapeutic agent that is required for a therapeutic dose is another example. The maximum concentration of therapeutic agent that can be tolerated without adverse effects may also be considered. These, and other, factors may be considered individually, or in combination, with each in determining the appropriate predetermined interval. In preferred embodiments, the biological half-life, minimum concentration and maximum concentration are considered collectively in establishing ideal predetermined intervals.

Parameters derived from factors intrinsic to an area of the brain targeted with the therapeutic agent, the brain itself, the overall CNS, or an overall medical state of a patient may also be considered in determining the predetermined interval. A measurement of the healing rate of a tissue in the target area of the brain may determine the length of the infusion interval because the reflux may be exacerbated by poor sealing of the tissue around the catheter. Other parameters include patient vitals (e.g., respiratory rate), results of MRI, CT scans, or the like.

The method may further include sealing tissue around the implanted intracranial catheter by designing an appropriate therapy regimen, which includes predetermining the infusion intervals. For example, a 28 day interval for 8 hour infusions for 5 consecutive infusions cycles could be optimal for a patient suffering from a lesion in one target area of the brain. Other intervals, such as a 32 day interval, might be more appropriate in other cases. In some cases, altering the intervals, infusion periods, and/or staggering the infusion cycles might be necessary based on changes in physical, mental or environmental conditions affecting the patient. Establishment of the appropriate predetermine intervals to affect the ideal therapy regimen is an important object of this disclosure.

The method may further include monitoring the target area of the brain before the first infusion and/or before or after each subsequent infusion to determine a value of a parameter. The method may also include determining the predetermined interval based on the value of the parameter. The predetermined interval of each infusion may be varied based on the value of the parameter(s) captured before or after each infusion. This allows medical professionals or automated decision or delivery systems to analyze relevant data and vary the therapy regimen based on methods disclosed herein.

The length of the infusion is not particularly limited by this disclosure and, like determining the predetermined interval of infusion, may be determined based on any one or more of a number of parameters disclosed herein or otherwise known in the art. The infusion may be administered for between about 4 and about 24 hours, especially for at least 5, 6, 7, 8, 9, 10, 11, 12, 13, 14, 15 or 16 hours and/or for less than 23, 22, 21, 20, 19, 18, 17, 16, 15, 14, 13, 12, 11, 10, 9 or 8 hours. Preferably, infusion is for about 8 hours. Infusion may also be administered for less than 4 hours or more than 24 hours based on factors unique to each patient or circumstance.

The therapeutic agent can be delivered by using an intracranial catheter having an internal diameter of from 0.3 mm to 0.5 mm. The therapeutic agent may be delivered via at least one chronically implanted intracranial catheter, especially an intraparenchymal catheter. The intracranial catheter may be a stepped catheter, i.e., having a cannula with a stepped outer diameter with the diameter of the step or steps decreasing from the proximal end to the distal end, such as those described in WO2007/024841. Alternatively the intracranial catheter may be a recessed step catheter such as those described in GB1213170.2, GB1215092.6 and GB1307921.5 all of which are incorporated herein by reference. Recessed step catheters comprise a distal section of tubing having an outer diameter that is smaller than the internal diameter of the catheter guide tube and arranged to create a recess for retaining brain tissue in the distal end section of the guide tube, between the guide tube and the distal section of tubing of the catheter. The retained brain tissue acts as a seal against reflux of fluid along the guide tube and catheter.

The therapeutic agent may be delivered via at least two chronically implanted intracranial catheters or via three or more of such catheters. Chronically implanted intracranial catheters refer to catheters that will be left in situ in the brain of a subject for at least six months, preferably for at least one year. The chronically implanted catheters may remain in place for the lifetime of a subject.

The method may further include reducing an inconvenience of the subject by leaving the at least one chronically implanted intracranial catheter in situ in the brain for at least about 6 months. Conventional intermittent infusion (e.g., 2 week intervals) of a therapeutic agent creates an inconvenience on patients and medical professionals alike beyond the physical and medical limitations discussed herein. Infusion regimens require multiple trips to infusion centers, hospital clinics, and the like, and reinserting the catheter for each infusion results in a negative impact on physical resources, costs and the physical and psychological well-being of the patient. It is well established that patient-centric medical methods are a significant aim in modern medicine. The longer predetermined intervals possible according to the methods and systems disclosed herein alleviate the negative impact, create a better overall patient experience, and present a significant advancement in modern medicine, particularly neuroscience.

The therapeutic agent may be selected from one or more of a chemotherapy drug, a neurotrophin, an enzyme, a growth factor, an antibody, an immunotoxin, small inhibitory RNA (siRNA), antisense oligonucleotides, drug releasing nanoparticles or a transgene. In preferred embodiments, the therapeutic agent is not a growth factor or carboplatin.

The therapeutic agent may be infused at a flow rate of 0.5 μl/min to 20 μl/min, preferably 1 μl/min to 10 μl/min, more preferably 2.5 μl/min to 5 μl/min. Volumes of about 1 ml to about 500 ml may be infused, preferably about 50 ml to about 300 ml, more preferably about 50 ml to about 150 ml may be infused. Preferably, about 100 ml of infusate is delivered.

The therapeutic agent may be administered in combination with aCSF. aCSF as disclosed herein may comprise glucose, proteins and ionic constituents. Preferably the aCSF comprises NaCl at a similar concentration to that found in natural CSF, that is to say the concentration is preferably within 15%, more preferably within 10% of the concentration in natural CSF. Preferably the aCSF comprises NaHCO3 at a similar concentration to that found in natural CSF, that is to say the concentration is preferably within 15%, more preferably within 10% of the concentration in natural CSF. Preferably the aCSF comprises KCl at a similar concentration to that found in natural CSF, that is to say the concentration is preferably within 15%, more preferably within 10% of the concentration in natural CSF. Preferably the aCSF comprises NaH2PO4 at a similar concentration to that found in natural CSF, that is to say the concentration is preferably within 15%, more preferably within 10% of the concentration in natural CSF. Preferably the aCSF comprises MgCl2 at a similar concentration to that found in natural CSF, that is to say the concentration is preferably within 15%, more preferably within 10% of the concentration in natural CSF. The aCSF can comprise glucose at a similar concentration to that found in natural CSF, that is to say the concentration is within 15%, preferably within 10% of the concentration in natural CSF. In preferred embodiments, the aCSF may omit glucose, so as to reduce the likelihood of bacterial growth in any catheter used to administer the composition to a subject. Most preferably, the aCSF does not comprise glucose or proteins.

The CNS disorders that may be treated by the methods and systems according to this disclosure include neurodegenerative diseases such as Alzheimer's disease, Huntington's disease, motor neuron diseases such as Amyotrophic Lateral Sclerosis (ALS), Multiple System Atrophy and Corticobasal degeneration, enzyme deficient conditions such as Tay Sachs Disease, Sandhoff Disease, Neuronal Ceroid Lipofuscinosis, Hunter Syndrome, Hurler disease and Gaucher's Disease, neuroinflammatory disease such as Multiple Sclerosis and Creutzfeldt-Jakob Disease, and acquired neurological injury such as stroke and traumatic brain injury. CNS disorders may also include cancer and depression for purposes of this disclosure. Methods and systems according to the disclosure may also be used in brain augmentation or improvement.

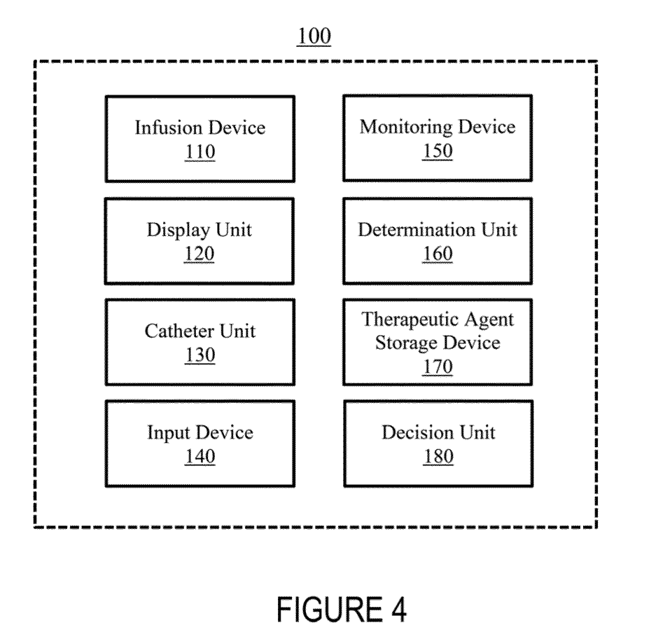

FIG. 4 illustrates a another embodiment. In this embodiment, there is provided a system 100 for reducing or preventing catheter reflux during infusion of a therapeutic agent into a target area of a brain of a subject. The system includes an infusion device 110 for administering a therapeutic agent to the target area of the brain, the infusion device 110 being any suitable device known in the art of neuroscience for infusing therapeutic agent into the mammalian brain. The infusion device 110 infuses therapeutic agent from a therapeutic agent storage device 170 through a catheter unit 130, such as at least one chronically implanted intracranial catheter, for delivering the therapeutic agent from the infusion device 110 to the target area. The infusion device 110 administers the therapeutic agent by intermittent infusion at the predetermined interval. The system 100 also includes a monitoring device 150 configured to monitor the target area of the brain after each infusion to determine a value of a parameter. The monitoring device 150 may include, for example, an MRI or CT scan, or other imaging or sensory devices, including fiber optic imaging systems, known in the art.

The system may further include a determination unit 160 configured to determine whether a predetermined interval is acceptable based on the value of the parameter and provide an answer to a medical professional on whether to carry out the infusions at the current predetermined intervals. For example, the determination unit may look for reflux and, if reflux is observed, decide that the period between infusions should be extended. The medical professional would then decide by how much to extend the period. In a case where the monitoring device monitors an observed reflux distribution pattern of a test infusate in the target area of the brain, the determination unit determines whether the observed reflux distribution pattern is sufficiently like a comparator reflux distribution pattern. The determination unit may be any suitable controller, such as a CPU or computer program executable on a CPU, configured to analyze data according to an algorithm or program and compute results to be output to any device or unit of system 100, such as a display unit 120 for displaying medical data to a medical professional.

The system may further include an input device 140 for allowing a medical professional to input information into the system 100 and control elements of the methods disclosed herein through the system 100. The input device 100 can be connected to the system via hardwiring or remotely through a network. Input device 100 may be, for example, a hand held device configured with touch screen controls.

The system may further include a decision unit 180 for calculating and executing controls based on inputs from other aspects of system 100. For example, the decision device may read parameters coming from the determination unit 160 coming from values recorded in the monitoring device 150, and make recommendations to the attending medical professional regarding the pre-infusion interval, the predetermined interval, the amount of therapeutic agent, the length of infusion, and any other factor relevant to determining a therapy regimen based on a specific patient or circumstance.

It will be appreciated that the above-disclosed features and functions, or alternatives thereof, may be desirably combined into different systems or methods. Also, various alternatives, modifications, variations or improvements may be subsequently made by those skilled in the art, and are also intended to be encompassed by the following claims. As such, various changes may be made without departing from the spirit and scope of this disclosure as defined in the claims.

1. A method of reducing or preventing catheter reflux during infusion of a therapeutic agent into a brain of a subject comprising administering the therapeutic agent to a target area of the brain by intermittent infusion at a predetermined interval.

2. The method according to claim 1, wherein the predetermined interval is at least about 21 days.

3. The method according to claim 1, wherein the predetermined interval is at least about 28 days.

4. The method according to claim 1, wherein the therapeutic agent is delivered by intracranial infusion.

5. The method according to claim 4, wherein the therapeutic agent is delivered by a intracranial catheter having an internal diameter of from about 0.3 mm to about 0.5 mm.

6. The method according to claim 4, wherein the therapeutic agent is delivered by at least one chronically implanted intracranial catheter.

7. The method according to claim 4, wherein the therapeutic agent is delivered by a recessed step intracranial catheter.

8. The method according to claim 1, wherein the therapeutic agent is delivered into gray matter of the brain.

9. The method according to claim 1, wherein the therapeutic agent is selected from one or more of a chemotherapy drug, a neurotrophin, an enzyme, a growth factor, an antibody, an immunotoxin, siRNA, antisense oligonucleotides, drug releasing nanoparticles or a transgene.

10. The method according to claim 1, wherein the therapeutic agent is not a growth factor or carboplatin.

11. The method according to claim 1, wherein the predetermined interval is at least about 35 days.

12. The method according to claim 1, wherein the predetermined interval is at least about 45 days.

13. The method according to claim 1, wherein the predetermined interval is at least about 6 months.

14. The method according to claim 6, further comprising sealing tissue around the at least one chronically implanted intracranial catheter.

15. The method according to claim 1, wherein the infusion is administered for between about 4 and about 24 hours.

16. The method according to claim 1, further comprising monitoring the target area of the brain after a first infusion to determine a value of a parameter; and
determining the predetermined interval based on the value of the parameter.

17. The method according to claim 16, wherein the parameter is a biological half-life of the therapeutic agent in the target area of the brain.

18. The method according to claim 16, wherein the parameter is a healing rate of a tissue in the target area of the brain.

19. The method according to claim 18, wherein during the first infusion, a test infusate is infused into the target area of the brain; and
the healing rate is determined using an observed reflux distribution pattern of the test infusate in the target area of the brain.

20. The method according to claim 19, further comprising:
establishing a comparator reflux distribution pattern for comparing the observed reflux distribution pattern; and
determining whether the observed reflux distribution pattern is sufficiently like the comparator reflux distribution pattern.

21. The method according to claim 19, wherein the therapeutic agent is infused into the target area of the brain during a second infusion at a time after the first infusion, the time based on the predetermined interval.

22. The method according to claim 19, wherein the test infusate and the therapeutic agent comprise one infusate infused during the first infusion;
the test infusate is front-loaded in the one infusate; and
the therapeutic agent is infused into the target area of the brain immediately following the test infusate.

23. The method according to claim 19, wherein the test infusate comprises:
a tracer; and
a carrier for carrying the tracer.

24. The method according to claim 19, wherein the volume of the test infusate is about 60 μl (microliters).

25. The method according to claim 16, further comprising varying the predetermined interval of each infusion based on the value of the parameter.

26. A method of treating a CNS disorder comprising administering a therapeutic agent to a target area of a brain of a subject by intermittent infusion at a predetermined interval.

27. The method according to claim 26, wherein the predetermined interval is at least about 21 days.

28. The method according to claim 26, wherein the predetermined interval is at least about 28 days.

29. The method according to claim 26, wherein the therapeutic agent is delivered by intracranial infusion.

30. The method according to claim 26, further comprising monitoring the target area of the brain after each infusion to determine a value of a parameter; and
determining the predetermined interval based on the value of the parameter.

31. The method according to claim 26, wherein therapeutic agent is delivered by at least one chronically implanted intracranial catheter.

32. The method according to claim 30, wherein the parameter is a biological half-life of the therapeutic agent in the target area of the brain.

33. The method according to claim 30, wherein the parameter is a healing rate of a tissue in the target area of the brain.

34. The method according to claim 30, further comprising varying the predetermined interval of each infusion based on the value of the parameter.

35. The method according to claim 26, further comprising reducing an inconvenience of the subject by leaving the at least one chronically implanted intracranial catheter in situ in the brain for at least about 6 months.

36. The method according to claim 26, further comprising increasing a therapeutic effectiveness of the therapeutic agent in the target area of the brain.

37. The method according to claim 26, wherein the therapeutic agent is not a growth factor or carboplatin.

38. The method according to claim 26, wherein the therapeutic agent is delivered by a intracranial catheter having an internal diameter of from about 0.3 mm to about 0.5 mm.

39. The method according to claim 26, wherein the therapeutic agent is delivered by a recessed step intracranial catheter.

40. The method according to claim 26, wherein the therapeutic agent is delivered into gray matter of the brain.

41. The method according to claim 26, wherein the therapeutic agent is selected from one or more of a chemotherapy drug, a neurotrophin, an enzyme, an antibody, an immunotoxin, siRNA, antisense oligonucleotides, drug releasing nanoparticles or a transgene.

42. A system for reducing or preventing catheter reflux during infusion of a therapeutic agent into a target area of a brain of a subject comprising:
an infusion device for administering a therapeutic agent to the target area of the brain;
at least one chronically implanted intracranial catheter for delivering the therapeutic agent from the infusion device to the target area;
a monitoring device configured to monitor the target area of the brain after each infusion to determine a value of a parameter;
a determination unit configured to determine whether a predetermined interval is acceptable based on the value of the parameter, wherein
the infusion device administers the therapeutic agent by intermittent infusion at the predetermined interval.

43. The system according to claim 42, further comprising a therapeutic agent storage device for storing the therapeutic agent during infusion.

44. The system according to claim 43, further comprising an input device for inputting an instruction to the system regarding a therapy decision.

45. The system according to claim 42, further comprising a display unit for displaying an element relevant to the predetermined interval.

46. The system according to claim 42, further comprising a decision unit for executing a control based on an output from at least one of the monitoring device and the determination unit.

47. The system according to claim 42, wherein the monitoring device monitors an observed reflux distribution pattern of a test infusate in the target area of the brain; and
the determination unit determines whether the observed reflux distribution pattern is sufficiently like a comparator reflux distribution pattern.

48. A therapeutic agent delivery system configured to reduce or prevent catheter reflux during infusion of a therapeutic agent into a target area of a brain of a subject, the system comprising:
an infusate including the therapeutic agent and a lead portion;
a catheter for delivering the infusate to the target area of the brain; and
an infusion controller configured to control the delivery of the infusate through the catheter, wherein
the lead portion includes a carrier and a tracer, the lead portion front-loaded in the catheter with respect to the therapeutic agent.

49. The therapeutic agent delivery system according to claim 48, wherein the carrier is aCSF.

50. The therapeutic agent delivery system according to claim 48, wherein the tracer is Gadolinium.

51. The therapeutic agent delivery system according to claim 48, wherein the volume of the lead portion is about 60 μl (microliters).
